SEQUENCE LISTING (2 sequences)

SEQ ID NO: 1 - DNA, 20 bp, Human immunodeficiency virus
aattaaccctcactaaaggg

SEQ ID NO: 2 - DNA, 20 bp, Human immunodeficiency virus
taatacgactcactataggg

(19) United States
(12) Patent Application Publication
(10) Pub. No.: US 2011/0020831 A1
(43) Pub. Date: Jan. 27, 2011

(54) GENETIC DETECTION OF HIV-1 STRAINS THAT USE THE CXCR4 CO-RECEPTOR

(75) Inventors: Chris Marlene Verhofstede, Mechelen (BE); Linos Peter Roger Vandekerckhove, Mechelen (BE); Lieven Jozef Stiuver, Mechelen (BE)

Correspondence Address: Patton Boggs LLP/ Johnson & Johnson, 8484 Westpark Drive, Suite 900, McLean, VA, 22102, US

(73) Assignees: GHENT UNIVERISTY, Ghent (BE); VIRCO BVBA, Beerse (BE)

(21) Appl. No.: 12/933,836

(22) PCT Filed: Mar. 20, 2009
(86) PCT No.: PCT/EP2009/053286

§ 371 Date: Sep. 21, 2010

(30) Foreign Application Priority Data
Mar. 21, 2008 (EP) 08102864.9

(51) Int. Cl. C12Q 1/68

(52) U.S. Cl. 435/6

(57) ABSTRACT

The invention relates to a method for obtaining clonal HIV-1 sequence information from a clinical isolate derived from an HIV-1 infected individual to guide highly active anti-retroviral therapy (HAART) comprising the following steps;
a) extracting cell-associated viral nucleic acid
b) amplifying said nucleic acid
c) obtaining clonal sequence information
d) interpreting the clonal sequence information and determining the ratio of the minority viral species by bio-informatics means
   whereby the ratio obtained is used to guide HAART.

Entry of the Human Immunodeficiency virus type 1 (HIV-1) into host cells requires binding of the viral envelope glycoprotein (gp120) with the cellular CD4 receptor and one of two co-receptors, CCR5 or CXCR4. Virus strains are classified as CCR5-tropic (R5), CXCR4-tropic (X4) or dual tropic (R5/X4) according to their coreceptor use. Long before the discovery of the co-receptors, the existence of two types of viruses, with distinct cytopathogenic properties in vitro, was observed. Viruses characterized by their ability to infect T-cell lines such as MT-2 cells and their capacity to induce the formation of syncytia were called syncytium-inducing (SI), viruses that were not able to grow on MT2 cells were called non-syncytium inducing (NSI) (1). Years later it was demonstrated that MT-2 cells express CXCR4 but not CCR5 so that nearly all viruses previously characterized as SI isolates are now considered to be X4 or dual tropic. Nearly all isolates previously considered to be NSI are now classified as R5. A switch from predominantly R5 strains to X4 strains occurs in about half of the individuals infected with HIV subtype B and is associated with an accelerated disease progression (2). Although X4 viruses can appear at a certain infection stage, they seem to coexistence with and not out compete the R5 strains.

Until recently the determination of coreceptor tropism remained mostly restricted to research activities, but with the recent introduction of the first coreceptor antagonist in clinical practice, assessment of the coreceptor tropism tends to become part of the routine clinical laboratory practices. The currently available coreceptor blocker, maraviroc (Pfizer) has a strict specificity for R5 tropic strains and sensitive tropism determination assays must allow excluding the presence of X4 strains before administration of this drug.

Today, the prediction of coreceptor use is based mainly on phenotypic recombinant assays such as Trofile™ (Monogram Biosciences, San Francisco, Calif., USA) and Tropism Recombinant Test (VIRalliance, Paris, France). Besides phenotypic assays, genotypic methods that allow to predict the coreceptor tropism based on the amino acid composition of the V3 loop of the HIV-1 envelope glycoprotein and bioinformatic tools, have been developed (3, 4). These genotypic methods are more accessible for routine laboratories but the opinions on their clinical usefulness are still divided (5, 6). The HIV-1 envelope gene can present a huge intra-patient variability and it is an intrinsic shortcoming of current population sequencing methods that they lack sensitivity for minority species. This might account for some of the observed discordances between the results of genotypic and phenotypic tropism prediction.

Effective antiretroviral treatment is only possible with the simultaneous administration of 3 different drugs (guidelines DHHS, EACS) so-called HAART (highly active anti-retroviral therapy). Due to the toxicity and adverse effects of many of these antiretrovirals a switch to a new class of antiretroviral can be required even if there are no indications of treatment failure or resistance.

Cross-toxicities among one class are common and they often rule out a whole class of drugs. The recent finding that most commonly prescribed antiretroviral drugs such as didanosine, abacavir, efavirenz and ritonavir boosted protease inhibitors may be associated with increased cardiovascular risk has fuelled the need for a switch to drugs that are better tolerated. The results of HIV trials on structured therapy interruption (e.g., the SMART study: strategies for management of anti-retroviral therapy) showed a negative outcome due to an increase in opportunistic infections (OI) and mortality and revealed that uncontrolled HIV-replication can, despite higher numbers of CD4+ T cells, place patients at an increased risk of OI/death. In the specific situation when there is a need for switching, chemokine receptor antagonists can thus far not be used as, even with the enhanced Trofile™ assay, a minimum viral load of 1000 copies/ml is needed before the necessary tropism test can be performed.

The NNRTI Nevirapine can not be initiated in patients on HAART with a good immunological recovery. According to the current guidelines (DHHS/EACS) nevirapine can only be started in woman with CD4<250 CD4/μL, in man with CD4<350. The NNRTI efavirenz can not be started in patients with a psychiatric history (depression, suicide).

Patients with skin and/or liver problems due to one NNRTI are discouraged to take the other available NNRTI.

PI intolerance (nausea, diarrhea) is a frequently observed cross-class intolerance. Although the prevalence of the gastrointestinal side effects of PI difference, the nausea and diarrhoea are still important side effects in most recent comparative double blinded clinical trials (Castle, Artemis).

Patients with dyslipidemia will increase their risk for cardiovascular diseases when taken PIs.

The administration of the NRTI tenofovir is advised against in patients with impaired kidney function. Patients with an HLA-B5701 positivity should not be started on the NRTI Abacavir, due to the increased risk of abacavir hypersensitivity reaction.

These are some examples of common side effects and tolerability problems that seriously limit the options to constitute an effective HAART regimen. Patients suffering from these side effects or toxicities might still be adherent to their treatment and show an undetectable viral load at the time when a decision about a treatment shift has to be taken.

Both genotypic and recombinant phenotypic assays use patient plasma-derived viral RNA for amplification of for instance the HIV-1 envelope gene and subsequent sequencing or construction of chimeric viruses. In the conventional MT2 assay on the other hand, MT2 cells were infected with virus that was isolated from the patient's peripheral blood mononuclear cells (PBMC). Whether R5 and X4 viruses are equally represented in both blood compartments is unknown. The dynamics of R5 and X4 viruses in plasma and PBMC is important to understand. Extensive clonal sequencing and the position specific scoring matrix (PSSM) algorithm were used to genotype the different quasispecies in both blood compartments.

Currently, a CCR5 antagonist can only be initiated in patients with a viral load of >1000 copies/ml. This is the amount of virus that is needed to be able to perform virus tropism analysis. There is an unmet need in the market to have a reliable method available based on viral DNA information to guide HAART. The ability to perform for instance a tropism analysis on the viral DNA isolated from infected cells complies with this need and opens the possibility of CCR5 antagonists to a much higher number of patients in need for new medications.

Maraviroc has been favourable evaluated in the Motivate studies in triple class experienced patients. As <50% of this patient group does harbour an R5 only virus, the use of Maraviroc is strongly limited in this experienced patient group. The use of the same chemokine receptor blocker in naïve patients (Merit trial) did initially not show a better outcome over efavirenz, but after re-analysis with the Enhanced Sensitivity Trofile, equivalence to efavirenz was demonstrated. Therefore there is a strong believe that this drug should be positioned early in treatment in patients which need a switch of medications for intolerance reasons. As mentioned above, this patient group on virological successful HAART therapy does have an undetectable viral load and a tropism determination based on DNA is of important value.

The recent FDA and EMEA approval of the first CCR5 antagonist for use in treatment experienced patients infected with R5 virus only, has forced a renewed interest in coreceptor tropism and stimulated efforts to develop easy to use methods for the determination CXCR4 using (X4) viruses. Currently the time consuming and costly recombinant phenotyping assays (e.g. Trofile) remain the standard. Several bio-informatic interpretation systems that allow predicting HIV-1 coreceptor use based on the sequence of the V3 loop, were developed, but there is currently no consensus about the quality of these assays and their use in clinical practice is discouraged (5). Studies aimed at comparing the results of genotypic assays with those of phenotypic methods all use sequence information obtained after-population sequencing of plasma virus. From earlier experiences with population sequencing for the detection of resistance mutations in the HIV-1 reverse transcriptase and protease, it is well known that current population sequencing assays have a detection limit for minority species detection in the range of 10-25% (assay dependent) and are therefore seen as lacking sensitivity for the detection of minority species. This might in part explain the lack of sensitivity of genotypic tropism assays for the detection of X4 virus as observed in some studies.

The current invention relates to high throughput clonal sequencing methods of part of the HIV-1 envelope gene in order to map the distribution of virus variants with different co-receptor tropism in both plasma and PBMC of selected individuals. These methods encompass any approach to obtain individual viral or proviral particle sequence information. Interpretation of the clonal sequences relied on the PSSM interpretation algorithm (3). Several reports have shown that this algorithm performs well (6). Moreover, PSSM is the only tool that gives a categorical output (X4/R5) as well as a continuous variable (referred to as the PSSM score).

The higher this score, the more similar the given V3 sequence is to an average actual X4 sequence.

It has now been shown that a clonal sequencing protocol, combined with the PSSM co-receptor prediction algorithm, allowed the detection of X4 strains in all of the 9 patients that were selected in this invention because of the ability of their virus to grow on the CXCR4⁺ CCR5⁻ MT2 cells. Additionally, X4 strains were detected in 2 of the 4 patients with indications for a progressed disease but no MT2 culture results available. More importantly, the results of clonal sequencing revealed that, even in a population of patients selected because of known X4 presence, the frequency of X4 strains in plasma remains relatively low (mean 22.6%; range 6.8 to 62.2%) with X4 viruses representing less than 10% of the total population in 5 of the 11 patients. Even if population sequencing protocols would allow detection of 10% minority species, it can be assumed from these data that the X4 strains would be missed in almost half of the patients tested here.

The invention relates to a method for obtaining clonal HIV-1 sequence information from a clinical isolate derived from an HIV-1 infected individual to guide highly active anti-retroviral therapy (HAART) comprising the following steps;
a) extracting cell-associated viral nucleic acid
b) amplifying said nucleic acid
c) obtaining clonal sequence information
d) interpreting the clonal sequence information and determining the ratio of the minority viral species by bio-informatics means
   whereby the ratio obtained is used to guide HAART.
Furthermore the invention concerns a method for obtaining clonal HIV-1 sequence information from a clinical isolate derived from an HIV-1 infected individual with undetectable viral load having toxic side effects ascribed to at least one antiviral used to guide HAART comprising the following steps;
   a) extraction cell-associated viral nucleic acid
   b) amplifying said nucleic acid
   c) obtaining clonal sequence information
   d) interpreting the clonal sequence information and determining the ratio of minority viral species by bio-informatics means
      whereby the ratio obtained is used to change the HAART, more specifically removing one antiviral compound from the HAART regimen and replacing it with another antiviral compound of the same class of antivirals or with another class of antivirals.
Part of the invention is also a method for obtaining clonal HIV-1 gp120 V3 loop sequence information from a clinical isolate derived from an HIV-1 infected individual to guide HAART comprising the following steps;
   a) extracting cell-associated viral nucleic acid
   b) amplifying the V3 loop sequence
   c) obtaining clonal V3 loop sequence information
   d) interpreting the clonal sequence information and determining the ratio of X4 tropic HIV-1 by bio-informatics means
      whereby the ratio obtained is used to guide HAART more specifically by using a co-receptor antagonist.
To the invention also belongs a method for obtaining clonal HIV-1 gp120 V3 loop sequence information from a clinical isolate derived from an HIV-1 infected individual with undetectable viral load having toxic side effects ascribed to at least one antiviral used to guide HAART comprising the following steps;
   a) extracting cell-associated viral nucleic acid
   b) amplifying the V3 loop sequence
   c) obtaining clonal V3 loop sequence information
   d) interpreting the clonal sequence information and determining the ratio of X4 tropic HIV-1 by bio-informatics means
      whereby the ratio obtained is used to change the HAART more specifically removing one compound from the HAART regimen and replacing it with another co-receptor antagonist.

A striking finding in accordance with the current invention was the significant higher representation of X4 strains in proviral PBMC DNA with X4 viruses representing a mean of 59.65% (range 7.1 to 94.4%) of the total virus population. In only 1 patient X4 viruses accounted for less than 10% of the virus population. In this later patient doubt exists about the accuracy of the X4 prediction since the PSSM scores of the clones that were classified as X4 are more indicative for dual tropism.

The possibility to use PBMC DNA instead of plasma RNA for a more sensitive detection of X4 viruses has some important advantage that might facilitate the implementation of genotypic tropism assays into routine laboratory practice. Cellular proviral DNA is easy to extract and stable. Amplification without a need for reverse transcription will reduce the complexity, cost and turn-around time of the procedure. Even more importantly, the use of PBMC will abrogate the need for a certain threshold viral load as a condition for the adequate tropism determination and allow patients to initiate a coreceptor blocking while on an active treatment regimen.

The possibility to recover both R5 and X4 viruses from peripheral blood leucocytes (PBMC) was expected as in vitro culture experiments have showed the possibility to culture viruses with SI and NSI properties from the same PBMC sample. Also in the latent reservoir of patients on HAART both R5 and X4 viruses have been detected by coculture and by sequencing (7). But information about the dynamics of R5 and X4 strains in both blood compartments was unavailable and the current invention of detecting a significant higher representation of X4 strains in PBMC compared to the plasma is surprising. As the coreceptor usage of an HIV-1 strain defines its cell tropism, it is to be expected that this coreceptor usage also modulates viral access to various human body compartments, due to tissue-specific cellular characteristics. The majority of HIV-positive individuals are initially infected with R5 HIV strains probably indicating a selective advantage of such variants for the cells present at the entry-sites. Gut-associated lymphoid tissue has been identified as the primary target of HIV-1 infection and in this rectosigmoid tissue expression of the CCR5 co-receptor on approximately 70% of CD4 T cells was demonstrated (8). In fresh peripheral blood leucocytes on the other hand, CXCR4 is expressed on a higher percentage of lymphocytes compared with CCR5 (mean 69.6% vs. 14.1%) (9). The high amount of CXCR4 expressing PBMC can explain why X4 viruses are more likely to infect these circulating CD4+ T cells. The bulk of free virus in the plasma probably derives from virus-production in lymphoid tissue and not in PBMC. Other factors might add to the different distribution. PCR amplification of HIV DNA in lymph node mononuclear cells and PBMC has demonstrated that the viral burden and replication in lymph nodes is 10-times higher than in PBMC (9). Schweighardt et al., 2004 also showed that the replication of R5 virus in primary CD4′ T-cell culture is more efficient than the replication of X4 virus in the same cells (10). A higher production of progeny virus by R5-infected CD4′ compared to X4-infected cells would add to the abundance of R5 viruses in the plasma.

The selection of patients included treated as well as untreated individuals. Results suggest that conventional antiretroviral regimens promote R5 virus over X4 virus (11). Three of the 13 patients that were studied were taken antiretrovirals at the time of sample collection. Though an influence of the treatment on the difference in distribution of X4 strains between plasma and PBMC in these 3 individuals can not be excluded, removal of these patients from the analysis does not change the final conclusion.

Due to practical constraints associated with the cost and workload of high throughput clonal sequencing, results are obtained on a relatively small number of patients. However, the fact that all but one patient showed the same tendency of over-representation of X4 strains in PBMC compared to plasma justify the above conclusion. Moreover, for both the one patient in whom a slightly higher representation of X4 was seen in plasma (patient 9532) and one of the 3 patient with a non significant difference between the X4 frequency in PBMC and in plasma (patient 9526), the maximum PSSM score remains low (respectively −6.96 and −4.52). These intermediate PSSM scores are more likely to indicate dual tropic virus instead of X4 tropic virus; Additionally, patient 9532 had a normal CD4 count at the time of sampling and the occurrence of X4 strains in patients with high CD4 counts of is very rare. Virus with a median PSSM score seem to have no preferential localisation in one of the blood compartments as was clearly illustrated after plotting the overall PSSM score distribution for plasma and PBMS. The same analysis revealed the pronounced location of strains with high PSSM scores in the PBMC compartment and this certainly deserves further study.

In conclusion, the invention shows that the frequency of occurrence of viruses classified as X4 based on the amino acid sequence of the envelope V3, is significantly higher in infected PBMC than in free plasma virus. These findings are important for future design of easy and sensitive assays for X4 virus detection.

Again, the CCR5 co-receptor antagonists are a new class of antiretroviral drugs. They are effective against viruses that use the CCR5 coreceptor but not against viruses that use the CXCR4 coreceptor. Before initiating the drug, the patients have to be screened for the presence of CXCR4 using virus strains. Current assays available for this screening lack sensitivity for minority species and need a threshold amount of free virus in the plasma to allow their performance. Based on the invention sequencing of the proviral DNA, extracted from PBMC or blood, is used as a more sensitive method for the determination of X4 presence in a patient. Besides increasing the sensitivity of the assay, this approach will facilitate the implementation of genotypic tropism testing in routine laboratory practice. Cellular proviral DNA is easy to extract and is a very stable molecule. Amplification without the need for reverse transcription will further reduce the complexity and also the cost and turn-around time of the procedure. Population sequencing of proviral DNA or a limited clonal sequencing is sufficient. Clinical studies are set up to define this more precisely, moreover, these studies must allow to evaluate the importance of minority X4 variants and the establishment of cut-off values for minority X4 variants with regard to the clinical efficiency of CCR5 antagonists.

The possibility to use proviral DNA also abolishes the need for a threshold amount of free circulating virus in the plasma. As a result, patients will be able to switch to the CCR5 inhibitor even if the virus replication is fully suppressed. This would allow switching to a CCR5 antagonist without the need for virological failure which is highly advantageous for the patient. Additional studies to define the evolution of X4 and R5 strains in the cellular reservoir of patients under suppressive medication are needed to evaluate the clinical usefulness of this approach.

The invention thus relates to the use of viral nucleic acids (RNA and/or DNA such as cytoplasm viral RNA or proviral DNA or nuclear viral RNA and proviral DNA obtained from PBMC, biopsy or tissues) for the determination of viral tropism, viral resistance and/or signature motif against anti-viral compounds (such as protease inhibitors, reverse transcriptase inhibitors, integrase inhibitors, maturation inhibitors, fusion or entry inhibitors or co-receptor antagonists) wherein the absence of a mutation and/or signature motif detection supports a therapy change to be decided by the physician.

The invention further relates to an in vitro method for the determination of the cellular co-receptor CXCR4 presence by extracting proviral nucleic acid (DNA) from PBMC or blood and sequencing said proviral nucleic acid where after a therapy change or switch for the patient can be decided allowing for instance switching to a CCR5 antagonist without the need for virological failure.

### DEFINITIONS

“HIV-1” denotes the human immunodeficiency virus type 1. Phylogenetic analysis of many isolates of HIV-1 from Africa and from other regions of the world have revealed three major lineages of HIV-1: viruses of group M (for main) are responsible for the majority of infections worldwide, group O (for outlier) is a relatively rare group currently found in Cameroon, Gabon, and France, and group N gathers non-M/non-O viruses. Within group M, in addition to subtype B, at least eight distinct non-B subtypes (A through H) and circulating recombinant forms (CRFs) have been proposed.

By “R5-tropic virus” is meant a HIV-1 virus which uses CCR5 co-receptor for entry into cells. By “X4-tropic virus” is meant a HIV-1 virus which uses CXCR4 co-receptor for entry into cells. By “R5/X4-tropic virus” is meant a HIV-1 virus which may use any of CCR5 and CXCR4 co-receptors for entry into cells.

“Primers” which are used for PCR amplification are oligonucleotides, i.e. nucleic acids generally of at least 12, preferably at least 15, and more preferably at least 20 nucleotides, and preferably no more than 30, more preferably no more than 40, still preferably no more than 50 nucleotides, which are hybridisable under high stringency conditions, and preferably complementary, to a region of double stranded HIV-1 cDNA molecule.

### FIGURES

FIG. 1. Distribution of X4 tropic viral strains in plasma-derived viral RNA and in PBMC derived proviral DNA clonal V3 sequences of 11 patients.

Significance p values are indicated on top of the columns

FIG. 2. Distribution of PSSM scores for the plasma-derived viral RNA and PBMC derived proviral DNA clonal sequences of the 10 patients in whom X4 tropic strains were detected.

In FIG. 3 (an illustration in longitudinal samples from the same patient) is shown that the analysis of proviral DNA from infected cells allows a more sensitive detection of CXCR4 tropic viruses than analysis of viral RNA from free plasma virus.

### EXAMPLES

### Example 1

### Methods for Extraction, Amplification, Sequencing and Tropism Prediction

Whole-blood samples were obtained in ethylenediamine tetraacetic acid (EDTA)-containing tubes. Plasma was separated by centrifugation and stored at −80° C. PBMC were recovered after Ficoll-Hypaque density centrifugation and 10⁷ cells were used immediately for virus culture. Remaining cells were stored in liquid nitrogen. Isolation of HIV was performed by cocultivation of the patient PBMC with 5×10⁶ pytohemagglutinin stimulated donor PBMC in RPMI 1640 medium supplemented with interleukin-2 as described. Cultures were considered positive and harvested after two consecutive positive p24 antigen determinations. One ml of the culture supernatant was transferred to a 5 ml culture of MT2 cells. Cells were checked visually for the presence of syncytia every two days. P24 antigen determination was performed on day 5, 10 and 20.

Plasma HIV-1 RNA was quantified with the Amplicor HIV Monitor test kit (Roche Diagnostics Systems, Basel, Switzerland) with a lower limit of detection of 50 RNA copies/ml. The CD4⁺ T cell count was performed by flow cytometry, using the FACScan cytofluorometer and the Cellquest software (Beckton Dickinson Mountain View, Calif., USA), on freshly drawn blood samples. Absolute CD4 T cells were expressed per microliter.

RNA was extracted from 256 μl of plasma using the automated QIAamp Virus BioRobot MDx extraction platform (Qiagen, Hilden, Germany). Elution was performed with 60 μl elution buffer. DNA was extracted directly from PBMC by QIAgen whole blood extraction (Qiagen, Hilden, Germany)

Starting from viral RNA, NH₂-V4 gp120 amplicons were generated by one step RT-PCR using primers Env-6210F (nt 6221-6245 on JR-CSF, Genbank accession number M38429) and Env-R3 (nt 7507-7527 on JR-CSF) and SuperScript™ III RT/Platinum® Taq High Fidelity (Invitrogen). Thermal cycling consisted of reverse transcription for 20 min at 57° C., followed by amplification comprising 2 min at 94° C.; 50 cycles of 15 s at 92° C., 30 s at 60° C. and 30 s at 68° C.; and a final elongation for 7 min at 68° C. For clonal analysis, reactions were performed in seven-fold and pooled.

Starting from proviral DNA the same procedure was used with omission of the reverse transcription step and using AccuPrimen™ Pfx DNA polymerase (Invitrogen).

NH₂-V4 gp120 amplicons were cloned in pCR4-TOPO vector and transformed into competent TOP10 E. coli cells according to the manufacturer's recommendations (TOPO TA Cloning® Kit, Invitrogen). Individual colonies were picked for further analysis using the QpExpression robot (Genetix Limited, Hampshire, United Kingdom). Starting from NH₂-V4-containing TOPO plasmids, NH₂-V4 gp120 amplification was performed using primers Env-6210F and Env-R3, and AccuPrime™ Pfx DNA polymerase (Invitrogen). Thermal cycling comprised 2 min at 94° C.; 35 cycles of 15 s at 94° C., 30 s at 53° C. and 1.5 min at 68° C.; and a final elongation for 10 min at 68° C. NH₂-V4 gp120 amplicons were purified either using the QiaQuick PCR purification or QiaQuick Gel Extraction kit (Qiagen).

Sequencing reactions were prepared using the BigDye Terminator cycle sequencing kit (Applied Biosystems, Foster City, Calif., USA) with primers T3 (5′-AATTAACCCTCACTAAAGGG-3′) and 17 (5′-TAATACGACTCACTATAGGG-3′). Thermal cycling consisted of 25 cycles of denaturation at 96° C. for 10 s, primer annealing at 50° C. for 5 s and elongation at 60° C. for 4 min. Sequencing products were run on an ABI3730xl automated sequencer. Sequence editing and contig assembly were performed using SeqScape v2.5 (Applied Biosystems).

This is just one example of obtaining clonal sequence information. There are many other methods that can be applied to obtain clonal sequence information, either by using cloning in prokaryotic organisms, or by applying pyrosequencing technologies.

The co-receptor tropism of all clones was predicted based on the V3 loop sequence using a position specific scoring matrix (PSSM) (http://ubik.microbiol.washington.edu/computing/pssm/). An advantage of PSSM is that it not only provides a categorical interpretation (X4/R5) but also a continuous variable PSSM The higher this PSSM score, the more closely the sequence resembles those of known X4 viruses

This is just one example to obtain a bio-informatic prediction of a clonal V3 loop sequence. There are other bio-informatics methods available to obtain a prediction, including but not limited to support vector machine technologies, linear model learning machines and others. In principle, any bio-informatics algorithm that can make a reliable prediction of the virus tropism deduced from a sequence can be used,

Nucleotide alignment was performed using the ClustalW algorithm of AlignX (Vector NTI Advance™ 9). The final alignments were imported into the Phylip package in which distances were calculated using DNADIST under the Felsenstein 81 model and the transition/transversion ratio set at 4.0. Mid-point rooted trees were created by using the neighbor-joining method.

Fisher's exact test was used for the comparison of X4 frequencies.

### Example 2

### Applying the Clonal Amplification and Sequencing Methods to Clinical Isolates

Eleven patients were selected, all males infected with a subtype B HIV-1 virus. For 8 of these patients a positive MT2 culture result was obtained for the samples included. For three patients the MT2 culture was not performed but the patients were selected based on viral and immunological indications for a progressed disease. Most blood samples were collected in the pre-ART area. Eight patients were treatment naive, 2 were treated (3TC monotherapy, AZT+ddC in 1). The CD4 count ranged between 22 cells/μl and 818 cells/μl (mean 177). HIV plasma viral load results were available for 10 patients: mean 4.97; range 3.84-6.38 log copies/ml.

Patient's characteristics and results of amplification and cloning reactions are shown in Table 1. After removal of occasional sequences with mixed nucleotides, stop codons or frame shifts, a total of 427 RNA sequences and 330 DNA sequences were retained from 11 patients. The mean number of clonal RNA sequences available per plasma RNA sample was 39 (between 22 and 44), the mean number of proviral DNA sequences available per PBMC sample was 30 (18 to 47).

### Example 3

### Tropism Prediction on Clonal Sequences

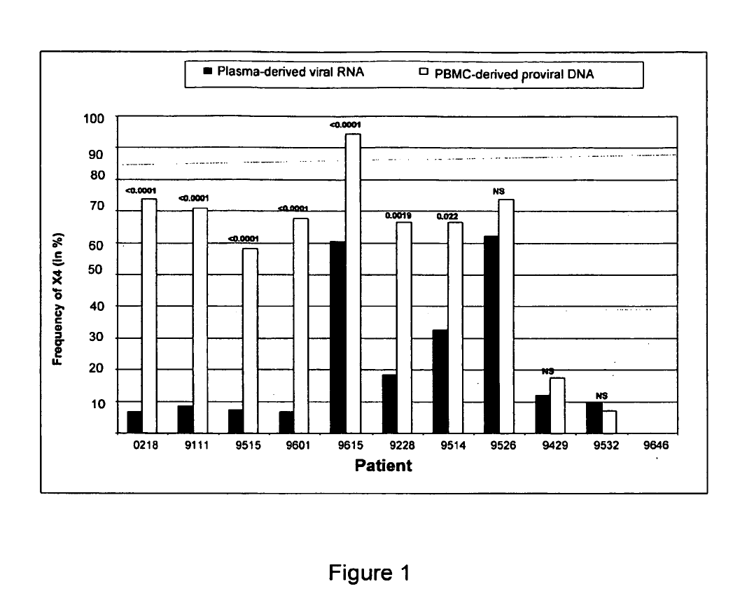

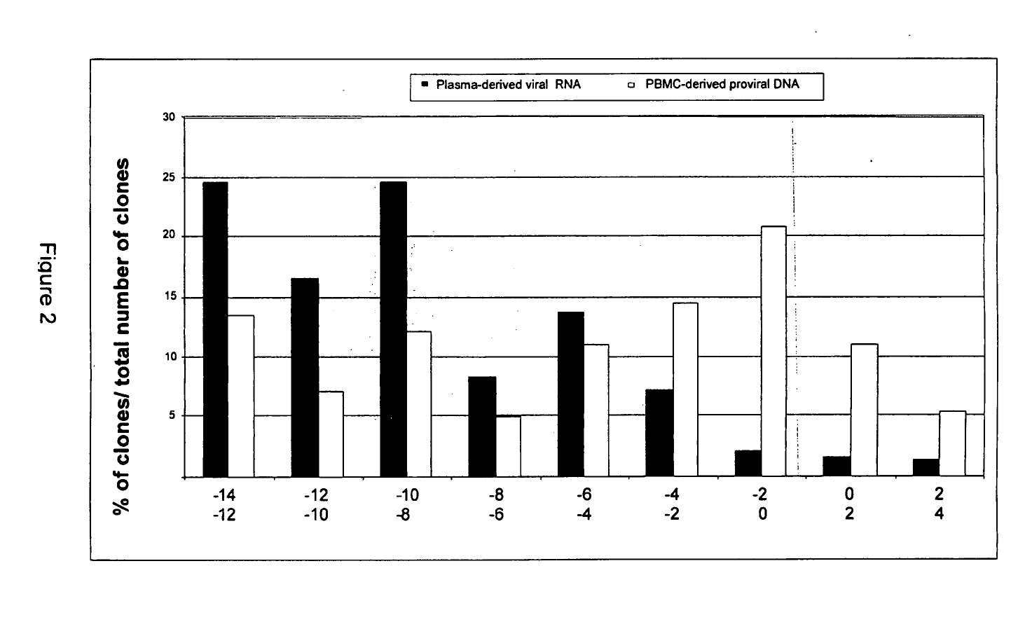

The position specific scoring matrix (PSSM) was used to deduce the coreceptor tropism of 427 V3 sequences. Results of the PSSM interpretation and the ratio of the PSSM scores that were obtained are printed in Table 1. An overview of the different V3 sequences with their frequency of occurrence and PSSM score is given in Table 2. Sequences predicted to be from X4 strains were detected in 10 of the 11 patients, including all patients with a positive MT2 culture and 2 of the 3 patients with other indications of disease progression. X4 strains were depicted from both plasma RNA and cellular DNA of all 10 patients, but the frequency of their occurrence was different for both blood compartments. Eighty four of the 427 plasma RNA sequences (19.7%) were scored as X4 versus 174 of the 330 PBMC DNA sequences (52.7%). On an individual patient level X4 variants were significantly more represented in PBMC compared to plasma (p<0.05) in 6 patients. A higher X4 representation in PBMC though not reaching statistical significance was observed in 3 patients. Only in one patient X4 strains were more represented in plasma compared to PBMC (9.5% vs 7.1%) but the difference remained low (FIG. 1).

### Example 4

### Distribution Differences of X4 Predicted Viruses

The higher abundance of X4 strains in PBMC was reflected in a higher mean PSSM score for DNA sequences but no difference in the range of the PSSM scores was observed between PBMC and plasma, indicating the coexistence of X4 and R5 strains with a broad diversity of PSSM scores in both blood compartments (see Table 2).

The only one patient with a higher X4 presence in plasma compared to cells (patient 9532) showed a narrow PSSM range for both the plasma and PBMC sequences and a low maximum PSSM score (−6.96). According to the PSSM algorithm, a score of −6.96 is more indicative for a dual-tropic virus than for a pure X4 strain. Of note, this patient also had a very high CD4 count (818 cells/μl).

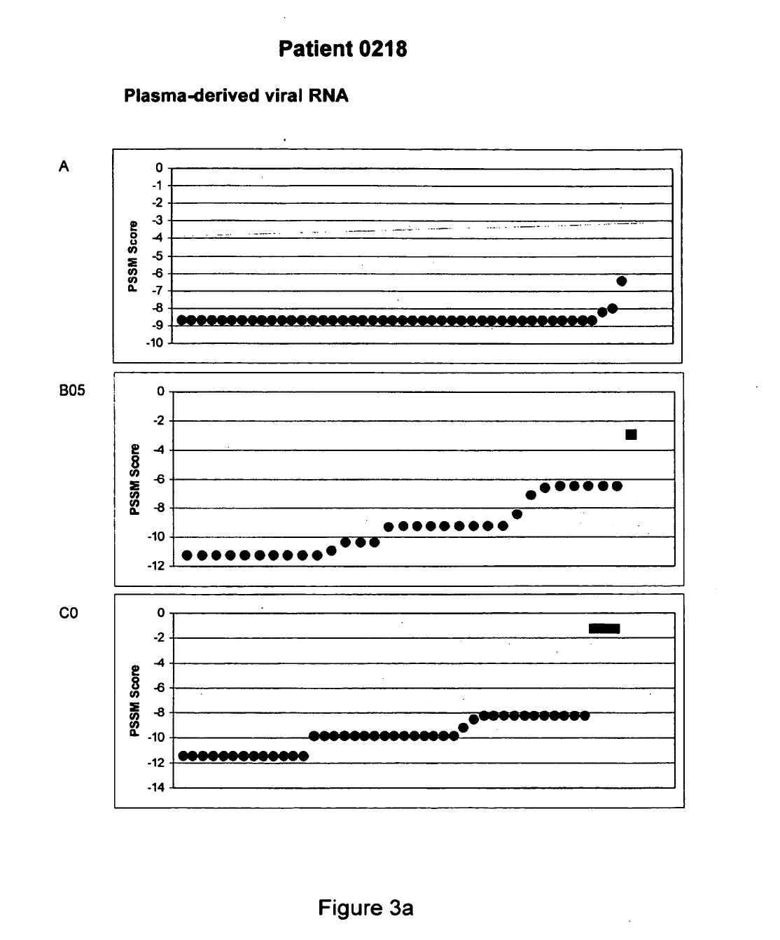

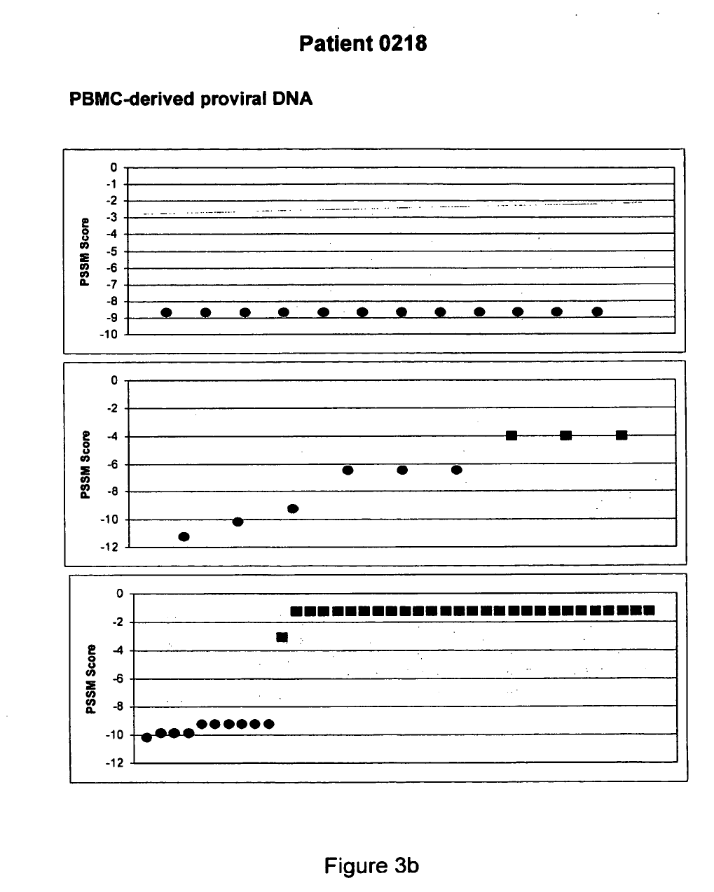

The overall distribution of PSSM scores in the plasma and PBMC sequences of the 10 patients with X4 viruses is graphically presented in FIG. 2. The results reveal the preferential localisation of strains with a PSSM score below −7.0 (indicating a close resembling with R5 viruses), in the plasma, while viral strain with a PSSM score higher than −3.0 (indicating a close resemblance with known X4 viruses) preferentially reside in the PBMC. Strains with a PSSM score between −7.0 and −3.0 seem evenly distributed in both blood compartments. These intermediate scores are likely to be associated with a dual-tropic phenotype.

In FIG. 3 (an illustration in longitudinal samples from the same patient) is shown that the analysis of proviral DNA from infected cells allows a more sensitive detection of CXCR4 tropic viruses than analysis of viral RNA from free plasma virus.

### Example 5

### Therapy Change in Patient with Undetectable Viral Load

Examples of common side effects and tolerability problems that seriously limit the options to constitute an effective regimen are provided hereunder.

Examples of drug ascribed side effects are given:

The aim of antiretroviral therapy is to maintain undetectable viral load both in ART naïve and highly experienced patients. Since the introduction of ART an important decrease in HIV associated mortality was observed in HIV infected patients. Age on itself is an important risk factor for cardiovascular disease which can involve all organs inclusive the kidney. Therefore clinicians facing a problem choosing the optimal NRTI background therapy as abacavir is associated with increased risk for myocardial infarction, and viread is associated with an increased risk of tubular kidney disease.
1. Patient 1: HIV-1 positive patient of 55 years old, HIV-1 positive since 13 years, father died on MI at the age of 52, was experiencing a depression when he lost his job 2 years ago, CD4 count 320CD4/μL, has undetectable VL since 8 years, is still smoking although stop smoking has been systematically advised over the last 4 years, has a LDL cholesterol of 210 mg/dl, BMI 32, type 2 diabetes, serum Creatinne 1.7 mg/dl, CD4 720, He is currently on abacavir/epivir fixed combination+atazanavir/ritonavir. The abacavir is doubling his risk for a myocardial infarction, and a switch towards an alternative regimen should be performed. Unfortunately tenofovir can not be advised taking in account the increased serum creatinine. The current used (FDA/EMEA approved) NRT's are not an option since CD4>350 (nevirapine) and the history of a depression (efavirenz). Maraviroc has a safe lipid profile and would be an option if tropism could be determinated and would reveal an R5 virus.
2. Patient 44 years old, seropositive and on a stable viread/epivir+efavirenz regimen since 4 years. The combination tablet Atripla is not yet available in his country. Patient initially started 5 years ago on viread/epivir+kaletra (fixed dose Lopinavir/ritonavir, but did not tolerate the kaletra (dyspepsia+diarrhea). The patient undertook a trip to Africa and forgot his Truvada in the first hotel. As his doctor always emphasises the importance of taking his tablets, he continued taking his efavirenz for 6 w in monotherapy. When the patient visited his doctor soon after his trip, his VL is 60000 copies/ml and his virus carried the K103N NNRTI signature mutation. The patient was reinitiated with viread/epivir boosted ATV and became soon undetectable but complained again from nausea and diarrhea. Switching towards maraviroc could be an option if the tropism determination would again reveal an R5 virus.

As discussed above patients suffering from these side effects or toxicities might still be adherent to their treatment and show an undetectable viral load. Making decisions about change in a treatment regimen and thereby removing the toxicity and hence the antiviral drug burden on the patients well being, can be guided by analysing the proviral nucleic acids and search for minority species viral sequences that would contain markers predicted to influence treatment outcome (presence of X4 tropic virus, or drug resistance mutations at low percentages).

The current invention therefore also relates to an in vitro method for the determination of the cellular co-receptor CXCR4 presence by extracting proviral nucleic acid (DNA) from PBMC or blood and sequencing said proviral nucleic acid where after a therapy change or switch for the patient can be decided allowing for instance switching to a CCR5 antagonist without the need for virological failure.

### REFERENCES

1. Asjo, B., J. Albert, A. Karlsson, L. Morfeldtmanson, G. Biberfeld, K. Lidman, and E. M. Fenyo. 1986. Replicative Capacity of Human-Immunodeficiency-Virus from Patients with Varying Severity of Hiv-Infection. Lancet 2:660-662.
2. Connor, R. I., K. E. Sheridan, D. Ceradini, S. Choe, and N. R. Landau. 1997. Change in coreceptor use correlates with disease progression in HIV-1-infected individuals. Journal of Experimental Medicine 185:621-628.
3. Jensen, M. A., F. S. Li, A. B. van't Wout, D.C. Nickle, D. Shriner, H. X. He, S. McLaughlin, R. Shankarappa, J. B. Margolick, and J. I. Mullins. 2003. Improved coreceptor usage prediction and genotypic monitoring of R5-to-X4 transition by motif analysis of human immunodeficiency virus type 1 env V3 loop sequences. Journal of Virology 77:13376-13388.
4. Sing, T., A. J. Low, N. Beerenwinkel, O, Sander, P. K. Cheung, F. S. Domingues, J. Buch, M. Daumer, R. Kaiser, T. Lengauer, and P. R. Harrigan. 2007. Predicting HIV coreceptor usage on the basis of genetic and clinical covariates. Antiviral Therapy 12:1097-1106.
5. Low, A. J., W. Dong, D. Chan, T. Sing, R. Swanstrom, M. Jensen, S. Pillai, B. Good, and P. R. Harrigan. 2007. Current V3 genotyping algorithms are inadequate for predicting X4 co-receptor usage in clinical isolates. Aids 21:F19-F26.
6. Gamido, C., V. Roulet, N. Chueca, N. Zahonero, A. Aguilera, K. Skrabal, E. Poveda, S. Carlos, F. Garcia, J. L. Faudon, C. de Mendoza, and V. Soriano. 2007. Evaluation of seven different bioinformatic tools to predict HIV-1 tropism in non-B subtypes and concordance with a phenotypic assay. Antiviral Therapy 12:S161-S161.
7. Delobel, P., K. Sandres-Saune, M. Cazabat, C. Pasquier, B. Marchou, P. Massip, and J. Izopet. 2005. R5 to X4 switch of the predominant HIV-1 population in cellular reservoirs during effective highly active antiretroviral therapy. Jaids-Journal of Acquired Immune Deficiency Syndromes 38:382-392.
8. Grivel, J. C., J. Elliott, A. Lisco, A. Biancotto, C. Condack, R. J. Shattock, I. McGowan, L. Margolis, and P. Anton. 2007. HIV-1 pathogenesis differs in rectosigmoid and tonsillar tissues infected ex vivo with CCR5- and CXCR4-tropic HIV-1. Aids 21:1263-1272.
9. Lee, B., M. Sharron, M. Tsang, M. M. Majka, M. Z. Ratajczak, L. J. Montaner, D. Weissman, and R. W. Doms. 1998. Quantification of CD4, CCR5, CXCR4, and STRL33 levels on differentially conditioned monocyte-derived macrophages, various subsets of peripheral blood leukocytes and CD34+ bone marrow progenitor cells. Blood 92:164a-164a.
9. Pantaleo, G., C. Graziosi, L. Butini, P. A. Pizzo, S. M. Schnittman, D. P. Kotler, and A. S. Fauci. 1991. Lymphoid Organs Function as Major Reservoirs for Human-Immunodeficiency-Virus. Proceedings of the National Academy of Sciences of the United States of America 88:9838-9842.
10. Schweighardt, B., A. M. Roy, D. A. Meiklejohn, E. J. Grace, W. J. Moreno, J. J. Heymann, and D. F. Nixon. 2004. R5 human immunodeficiency virus type 1 (HIV-1) replicates more efficiently in primary CD4(+) T-cell cultures than X4 HIV-1. Journal of Virology 78:9164-9173.
11. Delforge, M. L., C. Liesnard, L. Debaisieux, M. Tchetcheroff, C. M. Farber, and J. P. Vanvooren. 1995. In-Vivo Inhibition of Syncytium-Inducing Variants of Hiv in Patients Treated with Didanosine. Aids 9:89-90.

TABLE 1
Overview of patient characteristics and tropism prediction for plasma and PBMC samples

| | | | Time | | Viral | | | RNA clones from plasma | | | | |
|---|---|---|---|---|---|---|---|---|---|---|---|---|
| | | | since | | load | CD4 | | | | | Mean | |
| Patient | Collection | | diagnosis | Age | (log | cells/ | | | n X4/ | | PSSM | PSSM Score |
| ID | data | MT2 | (years) | (years) | c/ml) | (μl) | Therapy | n | n R5 | % X4 | Score | (range) |
| 0218 | 08/14/2006 | NA | 4 | 35 | 5.38 | 59 | no | 44 | 3/41 | 6.8 | −9.28 | [−11.45, −1.25] |
| 9111 | 1/08/1996 | pos | 8 | 45 | ND | 31 | 3TC | 36 | 3/33 | 8.3 | −12.16 | [−13.75, 3.27] |
| 9515 | 11/08/1995 | pos | 1 | 37 | 5.31 | 176 | 3TC | 41 | 3/38 | 7.3 | −11.72 | [−14.02, 3.05] |
| 9601 | 01/15/1996 | pos | 1 | 39 | >5.00 | 25 | no | 44 | 3/41 | 6.8 | −10.27 | [−11.61, 0.19] |
| 9615 | 5/06/1996 | NA | 6 | 27 | 4.72 | 118 | no | 38 | 23/15 | 60.5 | −5.21 | [−11.99, −0.79] |
| 9228 | 11/18/1996 | pos | 4 | 35 | 4.34 | 179 | no | 22 | 4/18 | 18.2 | −6.24 | [−11.48, 0.98] |
| 9514 | 6/03/1996 | pos | 1 | 35 | 3.84 | 338 | AZT + DDC | 40 | 13/27 | 32.5 | −7.23 | [−8.75, −1.67] |
| 9526 | 08/30/1995 | pos | 2 | 27 | 4.97 | 65 | no | 37 | 23/14 | 62.2 | −6.83 | [−8.35, −6.10] |
| 9429 | 1/11/1996 | pos | 8 | 49 | 5.47 | 326 | no | 42 | 5/37 | 11.9 | −8.18 | [−10.99, −0.86] |
| 9532 | 05/23/1996 | pos | 1 | 27 | 4.16 | 818 | no | 42 | 4/38 | 9.5 | −12.02 | [−12.73, −6.96] |
| 9305 | 03/23/1993 | pos | 1 | 33 | ND | 89 | no | 32 | 8/24 | 25.0 | | |
| 9633 | 12/10/1998 | NA | 11 | 29 | 6.38 | 22 | no | NA | NA | NA | | |
| 9646 | 11/13/1996 | NA | 1 | 44 | 5.15 | 51 | no | 41 | 0/41 | 0.0 | −12.33 | [−13.17, −10.74] |

| | | DNA clones from PBMC | | | | | | | |
|---|---|---|---|---|---|---|---|---|---|
| | | | | | Mean | PSSM | | | |
| Patient | Collection | | n X4/ | | PSSM | Score | Quality | | |
| ID | data | n | n R5 | % X4 | Score | (range) | control | p* | Rema [symbol] |
| 0218 | 08/14/2006 | 38 | 28/10 | 73.7 | −3.48 | [−10.19, −1.25] | ok | <0.0001 | |
| 9111 | 1/08/1996 | 31 | 22/9 | 71.0 | −2.29 | [−13.75, 3.27] | ok | <0.0001 | |
| 9515 | 11/08/1995 | 31 | 18/3 | 58.1 | −4.39 | [−13.74, 3.87] | ok | <0.0001 | |
| 9601 | 01/15/1996 | 34 | 23/11 | 67.6 | −2.97 | [−11.61, 1.12] | ok | <0.0001 | |
| 9615 | 5/06/1996 | 36 | 34/2 | 94.4 | −2.80 | [−11.99, −0.89] | ok | 0.0006 | |
| 9228 | 11/18/1996 | 21 | 14/7 | 66.7 | −1.86 | [−12.08, 3.17] | ok | 0.0019 | |
| 9514 | 6/03/1996 | 18 | 12/6 | 66.7 | −5.23 | [−8.75, 0.43] | ok | 0.022 | |
| 9526 | 08/30/1995 | 23 | 17/6 | 73.9 | −6.55 | [−8.35, −4.52] | ok | 0.41 | |
| 9429 | 1/11/1996 | 23 | 4/19 | 17.4 | −7.82 | [−12.91, −0.86] | ok | 0.7 | |
| 9532 | 05/23/1996 | 28 | 2/26 | 7.1 | −12.14 | [−13.24, −6.96] | ok | 1.0 | |
| 9305 | 03/23/1993 | NA | NA | NA | | | failed | | Resampling [symbol] |
| 9633 | 12/10/1998 | 22 | 0/25 | 0.0 | | [−12.05, −8.05] | failed | | RNA ampl [symbol] |
| 9646 | 11/13/1996 | 47 | 0/47 | 0.0 | −10.87 | [−12.97, −10.90] | | | R5 virus only |

*p value (Fisher's exact test) for the difference between X4 frequency in plasma and X4 frequency in PBMC [symbol]
indicates data missing or illegible when filed

TABLE 2
Listing of all different V3 loop amino acid sequences obtained
after clonal sequencing. The corresponding PSSM scores, PSSM
interpretation and frequency of occurence in plasma-derived viral
RNA (n RNA) and in PBMC-derived proviral DNA (n DNA) is indicated.
For each patient sequences are ranked according to the PSSM
score and the sequence with the lowest PSSM score is used as a
reference. X4 predictions are marked in bold.

| | 10 20 30 | PSSM | PSSM | | |
|---|---|---|---|---|---|
| | ....\|....\|....\|....\|....\|....\|....\| | Score | Pred | n RNA | n DNA |
| 0218_A | CTRPGNNTRKSIHIGPGSTFFATGAIIGDIRKAHC | −11.45 | R5 | 13 | 0 |
| 0218_B | .........RG..........T..E.......... | −10.19 | R5 | 0 | 1 |
| 0218_C | ..........G..............V.......Y. | −9.84 | R5 | 15 | 3 |
| 0218_D | .........RG..........T..E........Y. | −9.26 | R5 | 0 | 6 |
| 0218_E | .......A........................... | −9.16 | R5 | 1 | 0 |
| 0218_F | ..........G..............V....G..Y. | −8.47 | R5 | 1 | 0 |
| 0218_G | ..........G..............VV......Y. | −8.21 | R5 | 11 | 0 |
| 0218_H | ..........G..L..RRI....EK....T...Y. | −3.07 | X4 | 0 | 1 |
| 0218_I | ..........G..L..RRI....EK....TK.... | −1.25 | X4 | 3 | 27 |
| 9111_A | CTRPSNNTRKSINMGPGRAFYTTGDIIGDIRQAHC | −13.75 | R5 | 18 | 1 |
| 9111_B | .........................V......... | −13.25 | R5 | 1 | 0 |
| 9111_C | ....N.............................. | −13.24 | R5 | 11 | 6 |
| 9111_D | ............................N...... | −12.93 | R5 | 0 | 1 |
| 9111_E | ....D.............................. | −12.77 | R5 | 1 | 0 |
| 9111_F | ....N....................V......... | −12.74 | R5 | 2 | 1 |
| 9111_G | ....N...................K.ST.V.K.S. | 2.51 | X4 | 0 | 2 |
| 9111_H | ....HT..L.K.PI...K......R.ST...K.S. | 1.09 | X4 | 0 | 2 |
| 9111_I | ....HT..L.K.PI..........N.S—.K.K... | 1.21 | X4 | 0 | 1 |
| 9111_J | ....HT..L.K.PI..........Q.SA...K.S. | 1.87 | X4 | 2 | 12 |
| 9111_K | ....HT..L.K.PI...KT.....R.ST...K.S. | 2.39 | X4 | 0 | 1 |
| 9111_L | ....HT..L.R.PI........K.Q.SA...K.S. | 3.04 | X4 | 0 | 1 |
| 9111_M | ....HT..L.K.PI........K.Q.SA...K.S. | 3.27 | X4 | 1 | 2 |
| 9111_N | ....HT..L.R.PI..........K.ST...K.Y. | 3.79 | X4 | 0 | 1 |
| 9515_A | CTRPGNNTRKSIHMGWGRQFYATGEIIGDIRQAHC | −14.02 | R5 | 1 | 0 |
| 9515_B | .............L...................Y. | −13.74 | R5 | 0 | 2 |
| 9515_C | .................................Y. | −13.09 | R5 | 27 | 3 |
| 9515_D | ..........G......................Y. | −12.91 | R5 | 1 | 0 |
| 9515_E | .............................V...Y. | −12.42 | R5 | 1 | 0 |
| 9515_F | .........R.......................Y. | −12.32 | R5 | 6 | 4 |
| 9515_G | .........R........E.....A...N...... | −12.17 | R5 | 0 | 2 |
| 9515_H | .........R..................N....Y. | −11.49 | R5 | 1 | 0 |
| 9515_I | ..........................V......Y. | −11.46 | R5 | 1 | 0 |
| 9515_J | .....D.........................R.Y. | −10.51 | R5 | 0 | 1 |
| 9515_K | ........GQ..Y.....E.....A...N...... | −8.42 | R5 | 0 | 1 |
| 9515_L | ........GQ..Y.....RSH.SRA....V...Y. | −2.42 | X4 | 0 | 1 |
| 9515_M | ......R.GQ..Y.....R.H.SRA....VK..Y. | −0.35 | X4 | 0 | 2 |
| 9515_N | ......K.GQ..Y.....R.H.SRA....VK..Y. | 0.05 | X4 | 0 | 2 |
| 9515_O | ......K.GQ..Y.A...R.H.SKA....VK..Y. | 0.48 | X4 | 0 | 2 |
| 9515_P | ......K.GQ..Y.....R.H.SRA.L..VK..Y. | 0.98 | X4 | 0 | 5 |
| 9515_Q | ......K.GQ..Y..S..R.H.SRA....VK..Y. | 3.05 | X4 | 3 | 2 |
| 9515_R | ......K.GQ..Y.....R.H.SRA.L.GVK..Y. | 3.23 | X4 | 0 | 1 |
| 9515_S | ......K.GQ..Y..S..R.H.SRAV...VK..Y. | 3.55 | X4 | 0 | 2 |
| 9515_T | ......K.GQ..Y..S..R.H.SRA...NVK..Y. | 3.87 | X4 | 0 | 1 |
| 9601_A | CTRPNNNTRGSIPIGPGRAFYATGDIIGDIRQAHC | −11.61 | R5 | 0 | 1 |
| 9601_B | .........R......................... | −11.57 | R5 | 29 | 0 |
| 9601_C | .........R..T...................... | −9.74 | R5 | 12 | 7 |
| 9601_D | ......S..R..T...................... | −9.02 | R5 | 0 | 1 |
| 9601_E | .........R.........L....N.......... | −8.74 | R5 | 0 | 1 |
| 9601_F | .........R..T..-----............... | −8.44 | R5 | 0 | 1 |
| 9601_G | ......Y..RR.SM...K..RVK.N.......... | −0.17 | X4 | 0 | 1 |
| 9601_H | ......Y..RR.SM......RVK.N.......... | 0.19 | X4 | 3 | 21 |
| 9601_I | ......Y..RR.SM......RVK.N........Y. | 1.12 | X4 | 0 | 1 |
| 9615_A | CTRPSNNTRKSINIGPGRAFYTTGQIIGDIRQAHC | −11.99 | R5 | 1 | 0 |
| 9615_C | ............T....K...A............. | −10.07 | R5 | 1 | 0 |
| 9615_D | ............T........A............. | −9.71 | R5 | 9 | 1 |
| 9615_E | ............T...................... | −9.15 | R5 | 2 | 0 |
| 9615_F | ........K...T........A............. | −7.60 | R5 | 0 | 1 |
| 9615_G | ............T.....VL............... | −5.76 | R5 | 1 | 0 |
| 9615_H | ....N.....G.TM....VL...........R.Y. | −3.38 | R5 | 1 | 0 |
| 9615_I | ....N.....R.TM...GVW............... | −3.34 | X4 | 1 | 0 |
| 9615_J | ....N.....R.TM....VW............... | −3.06 | X4 | 14 | 8 |
| 9615_K | ....N.....R.TM....VL............... | −2.94 | X4 | 0 | 10 |
| 9615_L | ....N.....R.TM....VL........N...... | −2.12 | X4 | 5 | 8 |
| 9615_M | ....N.....R.TM....VL...........R... | −1.72 | X4 | 2 | 6 |
| 9615_N | ....N.....R.TM....VL........S...... | −1.39 | X4 | 0 | 1 |
| 9615_O | ....N.....R.TM....VL........N..R... | −0.89 | X4 | 0 | 1 |
| 9615_P | ....N.....R.TM....VL...........R.Y. | −0.79 | X4 | 1 | 0 |
| 9228_A | CTRPNNNTRKSIHIGPGRAFYATGEIIGNIRQAHC | −12.08 | R5 | 0 | 2 |
| 9228_B | ...L............................... | −11.48 | R5 | 5 | 2 |
| 9228_C | ...L...............L............... | −10.54 | R5 | 1 | 0 |
| 9228_D | ....Y...................QV.......F. | −7.36 | R5 | 0 | 1 |
| 9228_E | ....Y................T..AV.....K.S. | −6.44 | R5 | 4 | 1 |
| 9228_F | ....Y................T..TV..D..K.F. | −6.02 | R5 | 6 | 1 |
| 9228_G | ....Y...................QV.....K.F. | −5.65 | R5 | 2 | 0 |
| 9228_H | .......I..R............RR.Q.T.G.... | 0.98 | X4 | 0 | 2 |
| 9228_I | .......I..R............RR.E.T.G.... | 0.98 | X4 | 4 | 2 |
| 9228_J | .......I..R............RR.K.T.G.... | 2.08 | X4 | 0 | 5 |
| 9228_K | .......I..R............RG.K.T.G.... | 2.62 | X4 | 0 | 4 |
| 9228_L | .......I..R............KR.K.T.G.... | 3.17 | X4 | 0 | 1 |
| 9514_A | CTRPNNNTRKSIHIGPGRVFYATGEIIGDIRKAHC | −8.75 | R5 | 24 | 4 |
| 9514_B | ..........G........................ | −8.57 | R5 | 2 | 1 |
| 9514_C | ......................K............ | −7.35 | R5 | 1 | 0 |
| 9514_D | ......................A............ | −6.66 | R5 | 0 | 1 |
| 9514_E | ..........R.TL.......T............. | −4.43 | X4 | 12 | 6 |
| 9514_F | ..........R.TM.......T............. | −3.78 | X4 | 0 | 1 |
| 9514_G | ......S...R.TL.......T............. | −3.71 | X4 | 0 | 2 |
| 9514_H | ..........R.T........T............. | −3.32 | X4 | 0 | 2 |
| 9514_I | ..........R.TL.......T........K.... | −1.67 | X4 | 1 | 0 |
| 9514_J | ........K.R.TL.......T........K.... | 0.43 | X4 | 0 | 1 |
| 9526_A | CTRPNNNTGKSIHIGPGRAFHTTGQIIGDIRQAHC | −8.35 | R5 | 11 | 5 |
| 9526_B | ........RR..Y...................... | −6.61 | R5 | 2 | 0 |
| 9526_C | ...................S............... | −6.25 | R5 | 0 | 1 |
| 9526_D | ........RR..Y........A.DR.......... | −6.15 | X4 | 23 | 15 |
| 9526_E | ............................G...... | −6.10 | R5 | 1 | 0 |
| 9526_F | .A......RR..Y........A.DR.......... | −5.98 | X4 | 0 | 1 |
| 9526_G | ........RR..Y........A.DR......H... | −4.52 | X4 | 0 | 1 |
| 9429_A | CTRPNNNTRKSIHIGPGRAFYATGEIIGDIRQAHC | −12.91 | R5 | 0 | 2 |
| 9429_B | ....................H.............. | −11.24 | R5 | 0 | 2 |
| 9429_C | ....................H...A.......... | −10.99 | R5 | 1 | 0 |
| 9429_D | ....................H...D.......... | −10.93 | R5 | 1 | 3 |
| 9429_E | ....H...............H...D.......... | −10.46 | R5 | 2 | 0 |
| 9429_F | ....................H...D..E....... | −10.31 | R5 | 0 | 1 |
| 9429_G | ....................HV..A.......... | −10.06 | R5 | 4 | 2 |
| 9429_H | .......I............H...D........Q. | −9.56 | R5 | 2 | 0 |
| 9429_I | ....................H...D........Q. | −9.49 | R5 | 15 | 5 |
| 9429_J | ....................HV..A...N...... | −9.23 | R5 | 6 | 0 |
| 9429_K | .......I............H...D.......... | −7.40 | R5 | 1 | 0 |
| 9429_L | .I..................H...D........Q. | −5.95 | R5 | 2 | 3 |
| 9429_M | ..............R.R...H...D........QY | −5.58 | R5 | 0 | 1 |
| 9429_N | .......I.......G....H..RD.......... | −4.69 | R5 | 3 | 0 |
| 9429_O | ......KI.......G....H..RD.......... | −2.87 | X4 | 0 | 1 |
| 9429_P | .......I..R....G....H...D.......... | −2.68 | X4 | 3 | 0 |
| 9429_Q | .......I..R....G....H..RD.......... | −1.92 | X4 | 1 | 2 |
| 9429_R | ......KI..R....G....H...D.......... | −0.86 | X4 | 1 | 1 |
| 9532_A | CTRPSNNTRKGIHIGPGRAFYATGEIIGDIRQAHC | −13.24 | R5 | 0 | 3 |
| 9532_B | ....N.............................. | −12.73 | R5 | 33 | 19 |
| 9532_C | ....N...................A.......... | −12.48 | R5 | 1 | 0 |
| 9532_D | ....N.S............................ | −12.01 | R5 | 1 | 1 |
| 9532_E | ....N....R..............A.......... | −11.71 | R5 | 0 | 1 |
| 9532_F | ....N.....D.............A.......... | −10.76 | R5 | 3 | 1 |
| 9532_G | ....N.........E.......P............ | −9.36 | R5 | 0 | 1 |
| 9532_H | ....N.......Y.......I...K.......... | −6.96 | X4 | 4 | 2 |
| 9646_A | CIRPGNNTRKSIPMGPGRAFYATGDIIGNIRQAHC | −13.17 | R5 | 2 | 0 |
| 9646_B | .T...........I...........V..D...... | −12.97 | R5 | 3 | 24 |
| 9646_C | .T..N.......HI...Q.......V..D...... | −12.94 | R5 | 0 | 16 |
| 9646_D | .T...................T...V..D...... | −12.86 | R5 | 5 | 0 |
| 9646_E | .T...........I..................... | −12.64 | R5 | 1 | 0 |

| | | | | | |
|---|---|---|---|---|---|
| 9646_F | ...........V.I..........EV..D...... | −12.54 | R5 | 2 | 0 |
| 9646_G | ...........V.I...........V..D...... | −12.23 | R5 | 22 | 3 |
| 9646_H | .T...........I...........V......... | −12.14 | R5 | 1 | 0 |
| 9646_I | .V.........V.I...........V..D...... | −12.11 | R5 | 1 | 0 |
| 9646_J | .T...........I...........V..E...... | −11.98 | R5 | 0 | 3 |
| 9646_K | ...........V.I.......T..EV..D...... | −11.93 | R5 | 1 | 0 |
| 9646_L | ...........V.I.......T...V..D....Y. | −11.83 | R5 | 1 | 0 |
| 9646_M | .T...................T...V..D....Y. | −11.67 | R5 | 1 | 0 |
| 9646_N | .T..N........I...........V..D...... | −10.90 | R5 | 0 | 1 |
| 9646_O | ...........V.I.......T...V..D...... | −10.74 | R5 | 1 | 0 |

1. A method for obtaining clonal HIV-1 sequence information from a clinical isolate derived from an HIV-1 infected individual to guide highly active anti-retroviral therapy (HAART) comprising the following steps;
a) extracting cell-associated viral nucleic acid
b) amplifying said nucleic acid
c) obtaining clonal sequence information
d) interpreting the clonal sequence information and determining the ratio of the minority viral species by bio-informatics means
whereby the ratio obtained is used to guide HAART.

2. A method for obtaining clonal HIV-1 sequence information from a clinical isolate derived from an HIV-1 infected individual with undetectable viral load having toxic side effects ascribed to at least one antiviral used to guide HAART comprising the following steps;
a) extraction cell-associated viral nucleic acid
b) amplifying said nucleic acid
c) obtaining clonal sequence information
d) interpreting the clonal sequence information and determining the ratio of minority viral species by bio-informatics means
whereby the ratio obtained is used to change the HAART, more specifically removing one antiviral compound from the HAART regimen and replacing it with another antiviral compound of the same class of antivirals or with another class of antivirals.

3. A method for obtaining clonal HIV-1 gp120 V3 loop sequence information from a clinical isolate derived from an HIV-1 infected individual to guide HAART comprising the following steps;
a) extracting cell-associated viral nucleic acid
b) amplifying the V3 loop sequence
c) obtaining clonal V3 loop sequence information
d) interpreting the clonal sequence information and determining the ratio of X4 tropic HIV-1 by bio-informatics means
whereby the ratio obtained is used to guide HAART more specifically by using a co-receptor antagonist.

4. A method for obtaining clonal HIV-1 gp120 V3 loop sequence information from a clinical isolate derived from an HIV-1 infected individual with undetectable viral load having toxic side effects ascribed to at least one antiviral used to guide HAART comprising the following steps;
a) extracting cell-associated viral nucleic acid
b) amplifying the V3 loop sequence
c) obtaining clonal V3 loop sequence information
d) interpreting the clonal sequence information and determining the ratio of X4 tropic HIV-1 by bio-informatics means
whereby the ratio obtained is used to change the HAART more specifically removing one compound from the HAART regimen and replacing it with another co-receptor antagonist.

5. Use of a sequenced nucleic acid, preferably proviral nucleic acid, isolated from PBMC or blood of an HIV-1 infected patient with an undetectable viral load to guide or to decide a therapy change or switch for the patient.
